(19) United States
(12) Patent Application Publication
(10) Pub. No.: US 2002/0141977 A1
(43) Pub. Date: Oct. 3, 2002

(54) Immunotherapy based on dendritic cells

(76) Inventors: John Kevin Collins, County Cork (IE); Liam O'Mahony, Cork (IE)

Correspondence Address: JACOBSON HOLMAN PLLC, 400 SEVENTH STREET N.W., SUITE 600, WASHINGTON, DC, 20004, US

(21) Appl. No.: 10/113,569

(22) Filed: Apr. 2, 2002

(30) Foreign Application Priority Data
Apr. 2, 2001 (IE) 2001/0333

(51) Int. Cl. A61K 45/00; C12N 1/20; C12N 5/08

(52) U.S. Cl. 424/93.7; 435/368; 435/252.1

(57) ABSTRACT

Dendritic cells are exposed to at least one bacterial strain in particular bacterial species present in the human commensal flora. The bacterial strain may be a Lactobacillus and/or Bifidobacterium and/or salmonella strain. The exposed dendritic cells or a formulation, pharmaceutical or vaccine comprising such dendritic cells may be used in the prevention and/or treatment of various diseases such as inflammatory diseases.

### INTRODUCTION

The invention relates to dendritic cells.

Dendritic cells are professional antigen presenting cells specialised for the initiation of T cell immunity. Physical contact between dendritic cells and T cells is required for the induction of T cell immunity. Dendritic cells activate antigen-specific immune responses via two types of signalling steps. The first signal step involves the peptide-MHC/TCR interaction, while the second involves co-stimulatory molecules such as cell surface markers and cytokines.

Immune responses are characterised by their polarisation in the cytokines that are produced. Dendritic cells produce an array of cytokines when they present antigens to T cells thus influencing the cytokine microenvironment and subsequent immune response.

### STATEMENT OF INVENTION

The invention provides dendritic cells which have been exposed to at least one bacterial strain. The bacterial strain preferably has immunotherapeutic properties.

In a particularly preferred embodiment of the invention there is provided dendritic cells which have been exposed to bacterial species present in the human commensal flora.

In one embodiment the bacterial strain is a Lactobacillus, such as Lactobacillus salivarius, especially Lactobacillus salivarius subspecies salivarius and preferably Lactobacillus salivarius subspecies salivarius 433118.

In another embodiment the bacterial stain is a Bifidobacterium, such as Bifidobacterium infantis, especially Bifidobacterium infantis 35624.

In another embodiment the bacterial strain is salmonella, such as Salmonella typhimurium, especially Salmonella typhimurium UK1.

The dendritic cells may be exposed to dead bacteria, or components or mutants thereof.

The invention also provides an active derivative, fragment or mutant of dendritic cells of the invention.

In a further aspect the invention provides a formulation comprising dendritic cells of the invention or an active derivative, fragment or mutant thereof. In particular the invention provides a pharmaceutical comprising dendritic cells of the invention or an active derivative, fragment or mutant thereof. Also provided is a vaccine comprising dendritic cells of the invention or an active derivative, fragment or mutant thereof.

In a further aspect the invention provides a method for activating dendritic cells comprising exposing dendritic cells to at least one bacterial strain. The bacterial strain may be a strain as defined above.

The dendritic cells of the invention or an active derivative, fragment or mutant thereof may have anti-inflammatory properties and/or anti-cancer properties and/or immuno-regulatory properties. The dendritic cells of the invention or an active derivative, fragment or mutant thereof may enhance immunological tolerance of specific antigens and/or activate cell-mediated immune responses to specific antigens and/or activate humoral immune responses to specific antigens.

The dendritic cells of the invention or an active derivative, fragment or mutant thereof may stimulate regulatory T cell responses.

The bacteria used in the invention may establish distinct cytokine networks by maturing naive dendritic cells.

The dendritic cells of the invention or an active derivative, fragment or mutant thereof have potential therapeutic benefit in the following disease states: inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris.

A deposit of Lactobacillus salivarius strain 433118 was made at the NCIMB on Nov. 27, 1996 and accorded the accession number NCIMB 40829. The strain of Lactobacillus salivarius is described in WO-A-98/35014.

A deposit of Bifidobacterium infantis strain 35624 was made at the NCIMB on Jan. 13, 1999 and accorded the accession number NCIMB 41003. The strain of Bifidobacterium infantis is described in WO-A-00/42168.

A strain of Salmonella typhimurium UK1 is described by Wilmes-Risenberg et al., 1996, from whom a sample was obtained.

### DETAILED DESCRIPTION

This invention describes cytokine production by dendritic cells in response to different bacterial species, which influences the nature of subsequent T cell activation.

The microflora on mucosal surfaces are vast in number and complexity. Many hundreds of bacterial strains exist and account for approximately 90% of the cells found in the human body, the remainder of the cells being human. The vast majority of these bacterial strains do not cause disease and may actually provide the host with significant health benefits (e.g. bifidobacteria and lactobacilli). These bacterial strains are termed commensal organisms. Mechanism(s) exist whereby the immune system at mucosal surfaces can recognise commensal non-pathogenic flora as being different to pathogenic organisms.

The human immune system plays a significant role in the aetiology and pathology of a vast range of human diseases. Hyper and hypo-immune responsiveness results in, or is a component of, the majority of disease states. One family of biological entities, termed cytokines, are particularly important to the control of immune processes. Pertubances of these delicate cytokine networks are being increasingly associated with many diseases. These diseases include but are not limited to inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris.

The pre-programming of dendritic cells with bacteria would result in biologically active dendritic cells secreting regulatory cytokines. These regulatory cytokines subsequently stimulate controlling immune responses. This invention describes the potential of different bacterial strains in customising dendritic cell phenotype and function. In this way customisation of disease specific therapies may be accomplished using a selection of bacterial strains.

Recognition of bacterial species by dendritic cells results in distinct patterns of cytokine production and immune responses. The cytokines produced by dendritic cells are secreted into the extracellular milieu. These cytokines deliver an informative signal to the T cell interacting specifically with the dendritic cell. In addition, secreted cytokines will also interact with neighbouring cells not specifically interacting with the dendritic cell. This “bystander” effect results in many different cell types being influenced by the cytokine network established by bacterial stimulated dendritic cells.

Aberrant presentation of antigen by dendritic cells results in many disease states, such as autoimmune disease (Drakesmith et al., 2000). Thus, the re-establishment of immunological tolerance using appropriately primed dendritic cells is an attractive therapeutic option.

The immunomodulatory activity of dendritic cells has been demonstrated to have therapeutic potential in a number of model systems (Link et al., 2001). Dendritic cell mediated tolerance has been achieved in animal models of experimental autoimmune encephalomyelitis and spontaneous diabetes (Huang et al., 2000, Papaccio et al., 2000). The in vitro transfection of dendritic cells with cytokines, such as IL-10 and TGFβ, enhances their suppressive potential (Thorbecke et al., 2000) but gene therapy is still an inherently dangerous approach (Wilson, 2000). A more efficient and attractive approach would be to pulse dendritic cells in vitro with biologically active compounds which commit dendritic cells to an appropriate cytokine secretion pattern.

As the majority of cytokines may have both pro and anti-inflammatory activities, patterns or networks of cytokine release have been associated with different types of immune responses. The existence of T cells which differ in their pattern of cytokine secretion allows differentiation of inflammatory or immune responses into at least three categories, cell mediated or humoral responses or Th3/Tr1 regulatory responses. Th1 responses are categorised by INFγ, TNFβ and IL-2 production leading to a cell-mediated response while Th2 cells secrete IL-4, IL-5, IL-9, IL-10 and IL-13 resulting in a humoral response. Th3/Tr1 responses are characterised by T cell secretion of the regulatory cytokines IL-10 and TGFβ. Differentiation of T cells into either network depends on the cytokine milieu in which the original antigen priming occurs (Seder et al., 1992). In addition, activation of T cells by dendritic cells leads to their differentiation into distinct populations of effector cells differing in their cytokine secretion pattern (Mosmann & Sad, 1996). These primary immune responses may also be influenced by a number of other cell types including β T cells. Different types of stimulation may also direct this response such as immune complex deposition within inflammatory sites which increases IL-6 and IL-10 production and inhibits production of TNFα and IL-1β thus influencing the Th1/Th2 balance. For successful elimination of some pathogens, the correct cytokine network needs to be established, such as the intracellular bacterium Listeria monocytogenes which elicits a Th1 response while the extracellular parasite Nippostrongylus brasiliensis requires a Th2 response. Each of these T cell subsets produce cytokines that are autocrine growth factors for that subset and promote differentiation of naive T cells into that subset (for review see Trinchieri et al., 1996). These two subsets also produce cytokines that cross-regulate each other's development and activity. INFγ amplifies Th1 development and inhibits proliferation of Th2 T cells while IL10 blocks Th1 activation. While the molecular events controlling Th1 and Th2 development are poorly understood, specific dendritic cell subclasses have been demonstrated to influence the elucidation of these different responses (Maldonado-Lopez et al., 1999). Tr1 cells have a profound suppressive effect on antigen-specific T cell responses mediated by secretion of IL-10 and TGF□ (Groux et al., 1997) and cytokine independent mechanisms such as direct cell-cell contact. Stimulation of T cells by specific dendritic cells generates T cells that display the typical properties of Tr1 cells (Jonuleit et al., 2000).

The cytokine networks involved in immune responses are subject to a complex number of control pathways that normally result in restriction of cellular damage and eradication of the infectious organism. However, unregulated release of these cytokines can have damaging consequences. Incorrect Th1/Th2 responses may contribute to the pathogenesis of certain diseases. For instance, the healing form of leprosy (tuberculoid lesion) is associated with a Th1 response while uncontrolled leprosy (lepromatous lesion) is associated with Th2 responses. Chronic inflammatory responses can lead to the death of the host. For instance, rats infected with the protazoan parasite Trypanosoma brucei become cachectic, develop anaemia and eventually die. Production of the proinflammatory cytokines has been associated with the pathogenesis of many disorders. In Langerhans cell histiocytosis, cytokines may be involved in some of the tissue damage seen with this disease (Kannourakis & Abbas, 1994). Rheumatoid arthritis is a chronic inflammatory disease of the synovial joints resulting in cartilage destruction and bone erosion (Kouskoff et al., 1996). High levels of proinflammatory cytokines have been detected from patients with rheumatoid arthritis and these levels could be associated with disease activity, altered energy metabolism and food intake (Roubenoff et al., 1994). In patients with sepsis, cardiovascular shock and organ dysfunction may be initiated by the production of proinflammatory cytokines stimulated by the infectious organism particularly in patients with cerebral malaria (Kwiatkowski et al., 1990). Certain alleles of polymorphic sites associated with TNFα production have been shown to predict patients with cerebral malaria (McGuire et al., 1994) and severe sepsis (Stuber et al., 1996) who will be most adversely affected. Genetic predisposition to increased TNFα production may also be associated with the development of autoimmune diseases such as diabetes and systemic lupus erythematosus. Inhibition of proinflammatory cytokine production has reduced the damage caused by many disease states. IL-1RA reduces the severity of diseases such as shock, lethal sepsis, inflammatory bowel disease, experimental arthritis and proliferation of human leukaemic cells (for review see Dinarello, 1992). Inhibition of TNFα in septic shock prevents the syndrome of shock and tissue injury despite persistent bacteraemia in animal models. Loss of the TNF receptor type I in knockout mice protects against endotoxic shock (Pfeiffer et al., 1993). Anti-cytokine strategies in humans with sepsis have yielded disappointing results possibly due to complications such as the late administration of these factors after the initial inflammatory insult. However, studies involving neutralising TNFα antibodies in rheumatoid arthritis and Crohn's disease have had considerable success with significant reductions in disease activity being observed (Moreland et al., 1997, Stack et al., 1997). Inhibition of transcription factors, such as NF-κB, which are responsible for intracellular signalling in the inflammatory response have been successful in reducing tissue damage in animals with chronic intestinal inflammation (Neurath et al., 1996). Moreover, adoptive transfer of T cells secreting IL-10 inhibited colitis in a murine model (Asserman et al., 1999). Therefore, while the inflammatory response is critical to the defence and repair of host tissues, uncontrolled responses can result in significant tissue and organ damage and may result in the death of the host.

TGFβ refers to a family of closely related molecules termed TGFβ1 to -β5 (Roberts & Sporn, 1990). All are released from cells in a biologically inactive form due to their association with a latency protein which is believed to be a critical regulatory step. Three receptors have been identified for TGFβ. Only two of these receptors transduce an intracellular signal suggesting a decoy function for the third receptor. Like the MIP family, TGFβ also functions as a chemotactic factor for both monocytes and neutrophils. However, this cytokine has diverse effects as both pro and anti-inflammatory effects have been described. Aggregated platelets following vascular injury release TGFβ resulting in inflammatory cell recruitment to the tissue. Activated monocytes and neutrophils synthesize TGFβ further increasing cellular recruitment. Monocyte integrin expression is also enhanced by TGFβ as is the induction of collagenase type IV which may aid movement through basement membranes into inflammed sites (Wahl et al., 1993). TGFβ increases the expression of FcγRIII (CD16) which recognises antibody bound cells thereby increasing phagocytic activity. The production of inflammatory cytokines by monocytes can also be stimulated by TGFβ. However, expression of IL-1 receptor antagonist (IL-1RA) is also increased suggesting that this cascade, in part, may be self regulating. TGFβ is also important as a negative regulatory agent. It antagonises the effects of many of the inflammatory cytokines and inhibits the proliferation of thymocytes, B cells and haemapoietic stem cells. The activity of a number of cell types can be suppressed by TGFβ including natural killer (NK) cells, cytotoxic T lymphocytes and lymphokine activated killer (LAK) cells. TGFβ also has suppressive effects on the release of reactive oxygen and nitrogen intermediates by tissue macrophages (Ding et al., 1990). The immune inhibitory effects of TGFβ can most clearly be observed in its effects on diseases such as experimental arthritis, multiple sclerosis and graft rejection. Through the stimulation of matrix protein production, TGFβ may be important to wound healing which is also indicated by its chemotactic activity for fibroblasts (Roberts & Sporn, 1990). Therefore TGFβ may have important functions with regard to resolution of the inflammatory response and promotion of healing within the inflammatory lesion.

IL-4, like INFγ and IL-2, is a T cell derived cytokine. IL-4 has a molecular mass of 15 kDa and post-transcriptional glycosylation adds to this. While the IL-4 receptor can be membrane bound or secreted, they are coded for by separate genes unlike other soluble receptors which are derived by proteolysis of the membrane bound form. The effects of IL-4 seem to be species specific. This cytokine promotes murine macrophage proinflammatory cytokine synthesis while inhibiting production of the same cytokines in humans. IL-4 can enhance antigen-presentation (Aiello et al., 1990) and enhances T cell, B cell and mast cell proliferation (Arai et al., 1990). B cell class switching, MHC class II and FcεRII expression are all influenced by IL-4. IL-4 can also function as an anti-inflammatory agent. It can inhibit production of prostaglandins and collagenases (Corcoran et al., 1992). IL-4 may also promote apoptosis in stimulated monocytes (Mangan et al., 1992). IL-13 seems to be a cytokine that is functionally similar to IL-4, as both are T cell derived cytokines and both suppress monocyte proinflammatory cytokine production and affect surface antigen expression (Hart et al., 1995).

IL-10 is produced by T cells, B cells, monocytes and macrophages (De Waal Malefyt et al., 1991). This cytokine augments the proliferation and differentiation of B cells into antibody secreting cells (Go et al., 1990). IL-10 exhibits mostly anti-inflammatory activities. It up-regulates IL-1RA expression by monocytes and suppresses the majority of monocyte inflammatory activities. IL-10 inhibits monocyte production of cytokines, reactive oxygen and nitrogen intermediates, MHC class II expression, parasite killing and IL-10 production via a feed back mechanism (De Waal Malefyt et al., 1991). This cytokine has also been shown to block monocyte production of intestinal collagenase and type IV collagenase by interfering with a PGE₂-cAMP dependant pathway (Mertz et al., 1994) and therefore may be an important regulator of the connective tissue destruction seen in chronic inflammatory diseases.

IL-12 is a heterodimeric protein of 70 kD composed of two covalently linked chains of 35 kD and 40 kD. It is produced primarily by antigen presenting cells, such as macrophages, early in the inflammatory cascade. Intracellular bacteria stimulate the production of high levels of IL-12 (Ma et al., 1997). It is a potent inducer of INFγ production and activator of natural killer cells. IL-12 is one of the key cytokines necessary for the generation of cell mediated, or Th1, immune responses primarily through its ability to prime cells for high INFγ production (Schmitt et al., 1997). IL-12 induces the production of IL-10 which feedback inhibits IL-12 production thus restricting uncontrolled cytokine production. TGF-β also down-regulates IL-12 production (D'Andrea et al., 1995). IL-4 and IL-13 can have stimulatory or inhibitory effects on IL-12 production. Inhibition of IL-12 in vivo may have some therapeutic value in the treatment of Th1 associated inflammatory disorders, such as multiple sclerosis (Leonard et al., 1997).

Interferon-gamma (INFγ) is primarily a product of activated T lymphocytes and due to variable glycosylation it can be found ranging from 20 to 25 kDa in size. This cytokine synergizes with other cytokines resulting in a more potent stimulation of monocytes, macrophages, neutrophils and endothelial cells. INFγ also amplifies lipopolysaccharide (LPS) induction of monocytes and macrophages by increasing cytokine production, increased reactive intermediate release, phagocytosis and cytotoxicity (Donnelly et al., 1990). INFγ induces, or enhances the expression of major histocompatibility complex class II (MHC class II) antigens on monocytic cells and cells of epithelial, endothelial and connective tissue origin (Arai et al., 1990). This allows for greater presentation of antigen to the immune system from cells within inflamed tissues. INFγ may also have anti-inflammatory effects. This cytokine inhibits phospholipase A₂, thereby decreasing monocyte production of PGE₂ and collagenase (Wahl et al., 1990). INFγ may also modulate monocyte and macrophage receptor expression for TGFβ, TNFα and C5a thereby contributing to the anti-inflammatory nature of this cytokine. Probiotic stimulation of this cytokine would have variable effects in vivo depending on the current inflammatory state of the host, stimulation of other cytokines and the route of administration.

TNFα is a proinflammatory cytokine which mediates many of the local and systemic effects seen during an inflammatory response. This cytokine is primarily a monocyte or macrophage derived product but other cell types including lymphocytes, neutrophils, NK cells, mast cells, astrocytes, epithelial cells (Neale et al., 1995) endothelial cells and smooth muscle cells can also synthesise TNFα. TNFα is synthesised as a prohormone and following processing the mature 17.5 kDa species can be observed. Purified TNFα has been observed as dimers, trimers and pentamers with the trimeric form postulated to be the active form in vivo. Three receptors have been identified for TNFα. A soluble receptor seems to function as a TNFα inhibitor while two membrane bound forms have been identified with molecular sizes of 60 and 80 kDa respectively (Schall et al., 1990). Local TNFα production at inflammatory sites can be induced with endotoxin and the glucocorticoid dexamethasone inhibits cytokine production. TNFα production results in the stimulation of many cell types. Significant anti-viral effects could be observed in TNFα treated cell lines and the IFNs synergise with TNFα enhancing this effect (Wong & Goeddel, 1986). Endothelial cells are stimulated to produce procoagulant activity, expression of adhesion molecules, IL-1, hematopoitic growth factors, platelet activating factor (PAF) and arachidonic acid metabolites. TNFα stimulates neutrophil adherence, phagocytosis, degranulation, reactive oxygen intermediate production and may influence cellular migration (Livingston et al., 1989). Leucocyte synthesis of GM-CSF, TGFβ, IL-1, IL-6, PGE₂ and TNFα itself can all be stimulated upon TNFα administration (Cicco et al., 1990). Programmed cell death (apoptosis) can be delayed in monocytes (Mangan et al., 1991) while effects on fibroblasts include the promotion of chemotaxis and IL-6, PGE₂ and collagenase synthesis. While local TNFα production promotes wound healing and immune responses, the dis-regulated systemic release of TNFα can be severly toxic with effects such as cachexia, fever and acute phase protein production being observed (Dinarello et al., 1988).

Dendritic cell therapies for the treatment of cancer have achieved some success. However, a number of mechanisms have been described which allow tumour cells to escape immunological destruction. Although tumours express antigenic determinants they are not eliminated by the host's immune system. Either the antigens are not being presented efficiently and consequently do not elicit a powerful enough immune response or there is continuous selection, ongoing in the cancer patient, for tumour cells that can evade immune recognition. For efficient antigen presentation, the antigen needs to be expressed on professional antigen presenting cells (APC) through MHC class II to CD4 helper T cells and through MHC class I, on tumour cells, to CD8 cytotoxic T cells. This process also requires the interaction of co-stimulatory molecules such as B7-CD28, CD70-CD27 and CD40-CD40 complexes with appropriate cytokine production. In patients with cancer this system does not seem to operate effectively and this failure could be due to a number of reasons. The down-regulation of MHC molecules on tumour cells has been well described (Restifo et al., 1993) and the antigen processing machinery of the tumour cells may be defective (Cromme et al., 1994). Tumour cell antigen presentation in the absence of costimulatory molecules may induce tolerance as demonstrated by animal experiments where immune responses were amplified when B7-1 or B7-2 were expressed on tumour cells (Shu et al., 1997). The development of antigen-specific T cell anergy may be an early event in the tumour -bearing host, suggesting that tolerance to tumour antigens may represent a significant barrier to immunotherapy (Staveley-O'Carroll et al., 1998). However, tolerance to certain tumour specific antigens, such as carcinoembryonic antigen (CEA), may be broken by immunisation with a recombinant virus expressing CEA (Tsang et al., 1995). T cells that have been repeatedly activated express CD95 (Fas) on their surface and are therefore sensitive to killing by tumour cells expressing Fas ligand (Hahne et al., 1996). Thus, tumour cells could be inducing apoptosis in the T cells that are recognising them as foreign.

At initial stages of tumour growth in a murine model, anti-tumour immune responses are induced but with increasing tumour burden a generalised immunosuppression becomes evident (Gahan et al., 1997). Patients with advanced cancer are frequently found to exhibit impaired immune responses and a variety of immuno-suppressive mechanisms have been described. Usually, immuno-suppression is confined to the tumour region except for a few cases of advanced disease (O'Sullivan et al., 1996). Tumour derived products may interfere with the local immune response. Immuno-suppressive cytokines produced by tumour cells include transforming growth factor β (TGFβ), interleukin-10 (IL-10) and vascular endothelial growth factor (VEGF). These cytokines have a number of suppressive effects on tumour infiltrating lymphocyte function suggesting that potent immuno-suppressive mechanisms may be at work within the tumour bed (Spellman et al. 1996). IL-10 is also a potent inhibitor of tumour cytotoxicity by monocytes and alveolar macrophages. Prostaglandin production in the vicinity of the tumour inhibits IL-2 induced T cell proliferation while tumour cell induction of nitric oxide production decreased mononuclear cell proliferation. Immune suppressive factors in tumour bearing hosts may induce lymphoid apoptosis (O Mahony et al., 1993). Soluble antigens shed by tumour cells may interfere with immune responses to tumours. Host CD4 T cells may play a role in tumour immune evasion as induction of Th2 responses may inhibit Th1 cell-mediated responses which are thought to be important for anti-tumour immunity.

Vaccination with dendritic cells has been demonstrated to break immunological tolerance of tumour cells and induce tumour lysis via Th1 type responses. However, strategies to date have focussed on identifying specific tumour antigens and defining antigenic peptides that bind to the particular MHC alleles expressed by each patient (Nestle et al., 1998). A more general approach would be to use dendritic cells previously exposed to specific bacterial stimuli. Exposure to the bacterial strains outlined in this invention would activate dendritic cells in a manner appropriate for stimulation of anti-tumour immune responses irrespective of the antigens present. Dendritic cells could also be pulsed with tumour antigens in vitro or in vivo. Cytokine production by activated dendritic cells in the tumour microenvironment would promote anti-tumour immune responses.

The majority of pathogenic organisms gain entry via mucosal surfaces. Efficient vaccination of these sites protects against invasion by a particular infectious agent. Oral vaccination strategies have concentrated, to date, on the use of attenuated live pathogenic organisms or purified encapsulated antigens (Walker, 1994). However, vaccination with antigen-pulsed dendritic cells, previously exposed to biotherapeutic compounds, such as bacteria, could result in a more effective protective immune response.

The invention will be more clearly understood from the following examples.

### EXAMPLE 1

### Cytokine Profiles of Murine Bone Marrow Derived Dendritic Cells Stimulated with Probiotic and Pathogenic Bacterial Strains

### Method

Mice were sacrificed by cervical dislocation and long bones excised. All adherent connective and muscle tissue was removed. Bones were sterilized by a rapid immersion in 70% ethanol and rinse in sterile PBS. The marrow was flushed repeatedly from the bones using 3 ml HBSS per bone. The cells were pelleted and resuspended in sterile water to lyse RBCs. The cells were immediately resuspended in HBSS and centrifuged again. The cells were resuspended in 3 ml RPMI 1640 plus 150 μl of each antibody directed against B cells (ATCC, TIB229), anti Ia (ATCC, TIB150), anti-CD8 (ATCC, TIB 207) and anti-CD4 (ATCC, TIB 146). Following the addition of 50 μl of complement (Sigma) the cells were incubated @ 37° C. for 1 hour. Cells were washed twice and resuspended in 36 ml RPMI. 3 ml of cells per well were plated in a 12 well plate (Costar) and incubated overnight @ 37° C. The non-adherent cells were removed and a new 12 well plate (Costar) plated. 4 ng/ml IL-4 (R&D Systems) and 2 ng/ml GM-CSF (R&D Systems) were added. The cells were allowed to mature for 7-8 days @ 37° C. 1.5 ml of fresh medium was added to each well on day 4. Following maturation of these dendritic cells, cells were scraped off the plates, pooled and counted. Cells were typically re-plated at 5×10⁵/ml in one ml in a 24 well plate (Costar). Cells were stimulated with 10 μg/ml LPS (Sigma, L3024), bacteria (10²−10⁶ cells/ml) or remained non-stimulated. Following 24 hours of culture, supernatants were harvested, aliquoted and stored at −20° C. Culture supernatants were examined for IL-4, IL-10, IL-12, INFγ, TGFβ and TNFα levels using ELISAs (Pharmingen).

### Results

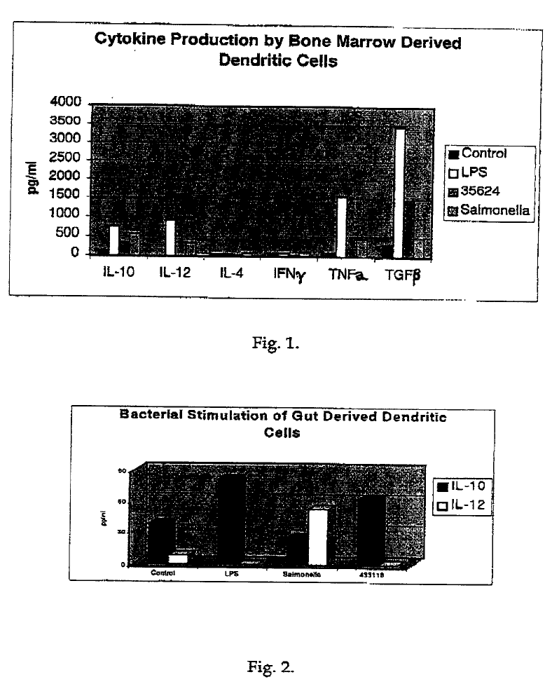

IL-4, IL-10, IL-12, INFγ, TNFα and TGFβ from dendritic cell culture supernatants were quantified following exposure to LPS, Bifidobacterium 35624 or Salmonella typhimurium (FIG. 1). LPS stimulated the production of IL-10, IL-12, TNFα and TGFβ compared to control cultures. Bifidobacterium 35624 enhanced the production of IL-10 and TGFβ, with a low level of TNFα stimulation. Salmonella typhimurium enhanced the production of IL-4, IL-10, IL-12, INFγ and TNFα, with a low level of TGFβ stimulation.

### EXAMPLE 2

### Cytokine Profiles of Murine Gastrointestinal Tract Derived Dendritic Cells Stimulated with Probiotic and Pathogenic Bacterial strains

### Method

Mice were anaesthetised and sacrificed by cervical dislocation (n=4). The gastrointestinal tract was removed, opened longitudinally and surface sterilised by a rapid immersion in 70% ethanol. The gastrointestinal tissue was incubated for 20 minutes shaking @ 37° C. in 25 mls HBSS containing DTT (0.145 mg/ml) and EDTA (0.37 mg/ml). Supernatants were decanted and the remaining tissue was incubated for 90 minutes shaking @ 37° in 25 mls RPMI containing collagenase (0.15 mg/ml) and DNAse (0.1 mg/ml). Supernatants were decanted and low speed centrifugation removed tissue debris and clumps of cells. Following high speed centrifugation, single cells were isolated. These cells were incubated with 10% normal mouse serum and magnetic CD11c beads for 15 minutes @ 4° C. Cells were passed through a magnetic column twice in order to enrich for CD11c positive cells. These cells were incubated for 24 hours with Lactobacillus 433118, or Salmonella typhimurium, or LPS or remained non-stimulated as a negative control. Supernatants were collected and stored at −70° C. IL-10 and IL-12 cytokine levels were quantified using ELISAs (Pharmingen).

### Results

Gut derived dendritic cells were incubated with a variety of bacterial stimuli (FIG. 2). Control cultures spontaneously produced IL-10 and IL-12. Stimulation with LPS enhanced IL-10 production but decreased IL-12 levels. Co-incubation with the Salmonella strain did not significantly alter IL-10 levels but did result in significant stimulation of IL-12 production. The probiotic 433118 enhanced the production of IL-10 and reduced IL-12 secretion.

### EXAMPLE 3

### Modulation of Cytokine Production in Bacterial Stimulated, Human Mesenteric Lymph Node Derived, Dendritic Cells

### Method

Following surgical removal of human colons, mesenteric lymph nodes were removed. Mesenteric lymph node cells were isolated using density gradient centrifugation and dendritic cells were purified using magnetic bead isolation. Dendritic cells were stimulated in vitro with Bifidobacterium 35624, Lactobacillus salivarius 433118 or Salmonella typhimurium for 3 days. Supernatants were removed and cytokines were quantified using ELISAs.

### Results

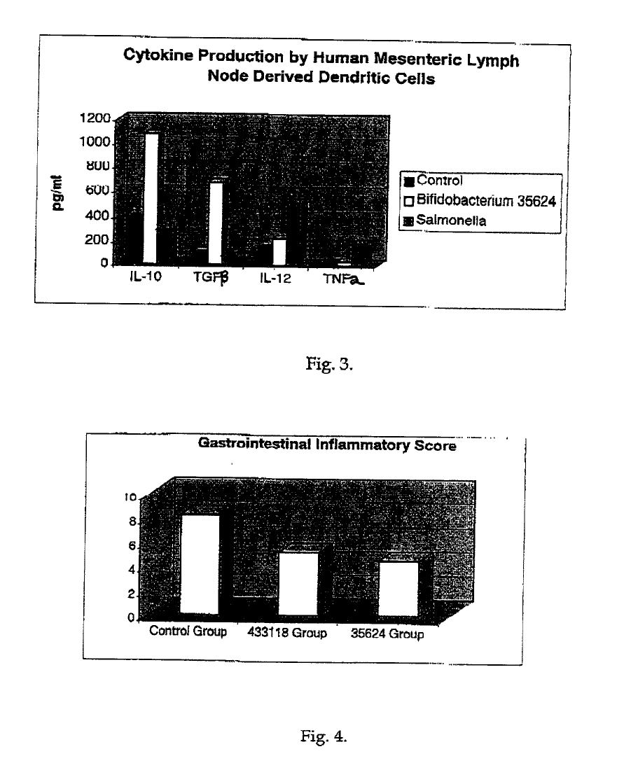

Dendritic cells stimulated with different bacteria secreted distinct cytokine profiles (FIG. 3). Bifidobacterium 35624 and Lactobacillus 433118 stimulated the production of Th2 and Th3 regulatory cytokines while Salmonella stimulated the production of Th1 regulatory cytokines. Lactobacillus 433118 was also found to stimulate the production of Th2 and Th3 regulatory cytokines (results not shown).

Dendritic cells isolated from both mice and humans react in a similar manner to bacterial stimulation. Thus, the use of murine models to examine the therapeutic potential of bacterial stimulated dendritic cells is appropriate.

### EXAMPLE 4

### Systemic Modulation of Immune-responsiveness Following Oral Consumption of Probiotic Bacteria

### Method

A feeding trial involving 3 groups (n=10/group) of IL-10 knockout mice was performed. Each group consumed the probiotic Lactobacillus 433118, or Bifidobacterium 35624 or a placebo product for 19 weeks. At this time point all mice were sacrificed by cervical dislocation. The gastrointestinal tract was removed, examined and graded histologically for inflammatory activity. Whole spleens were aseptically removed and the mononuclear cell population was isolated using mechanical disruption and density gradient centrifugation. 1×10⁶ spleen cells were stimulated in vitro with the probiotic 433118, or 35624, or the proinflammatory bacterium Salmonella typhimurium UK1, or remain non-stimulated as negative controls Following 72 hours of incubation, supernatants were harvested and stored at −70° C. ELISAs were subsequently performed in order to quantify IL-12, INFγ, TNFα and TGFβ cytokine levels (Pharmingen). Statistical analysis of group differences was performed using ANOVA analysis of variance.

### Results

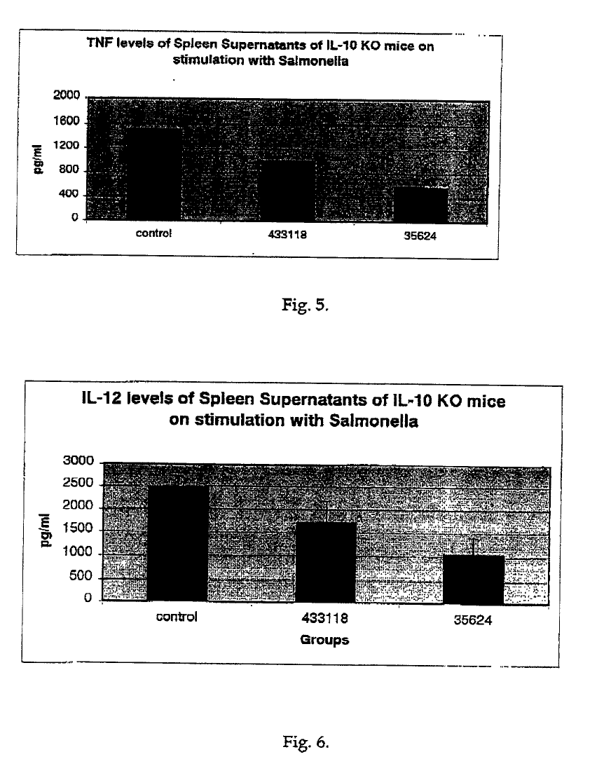

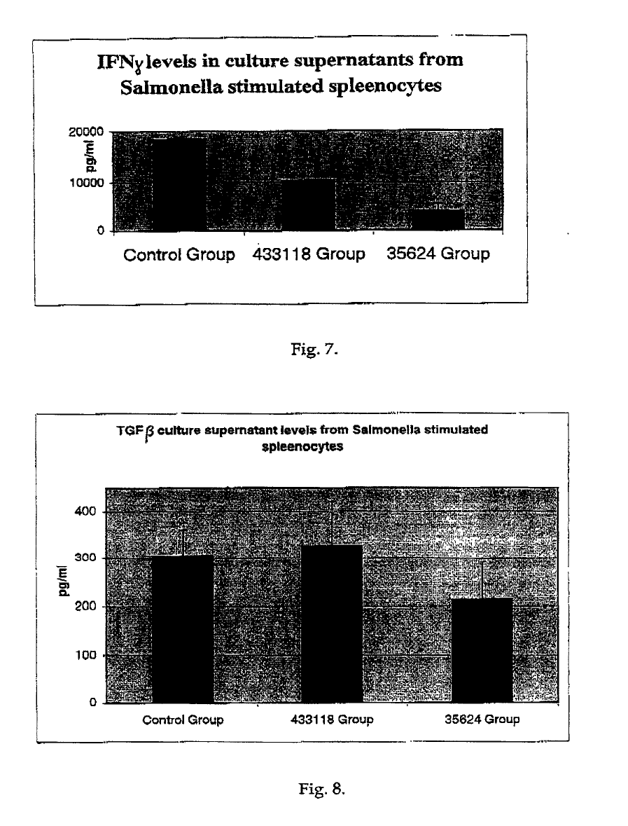

Significant numbers of both probiotic strains were recovered over the feeding trial period. Bifidobacterium 35624 was recovered at approximately 1×10⁵ CFU/g while Lactobacillus 433118 was recovered at approximately 1×10⁷ CFU/g. Gastrointestinal inflammatory scores were significantly reduced for the mice consuming either probiotic compared to the control group (FIG. 4). Following the in vitro stimulation of murine spleenocytes, significant decreases were observed for TNFα (FIG. 5), IL-12 (FIG. 6) and INFγ (FIG. 7) levels, but not TGFβ levels (FIG. 8).

This study demonstrates that an immunomodulatory signal was transmitted from the gastrointestinal tract, following consumption of the probiotic strains 433118 and 35624, to the spleen. Interaction of the consumed bacterial strains in the gastrointestinal tract with dendritic cells and subsequent migration of these dendritic cells to distant sites, such as the spleen, resulted in a significant alteration of cytokine production at these sites. It can be envisaged that administration of bacterial treated dendritic cells alone would also deliver this therapeutic immunological signal.

### EXAMPLE 5

### Systemic Anti-inflammatory Effects of Lactobacillus 433118 and Bifidobacterium 35624

### Method

DBA1 mice were fed with Lactobacillus 433118 or Bifidobacterium 35624 (n=10 per group). Following probiotic feeding, rheumatoid arthritis was induced following collagen tail vein injection in groups 2-4. Inflammatory arthritis was measured by quantifying footpaw swelling with callipers.

Group 1: Healthy mice—no interventions

Group 2: Placebo feed

Group 3: Lactobacillus 433118

Group 4: Bifidobacterium 35624

### Results

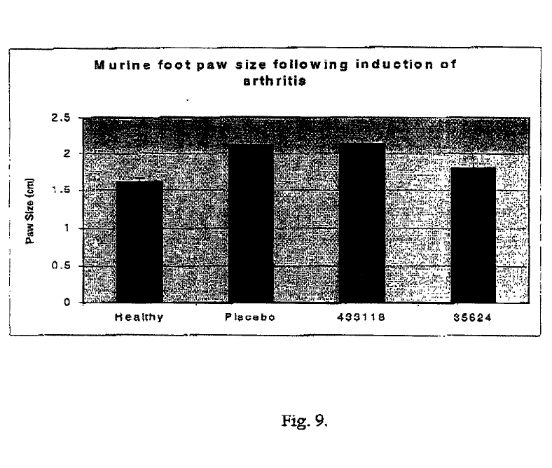

Footpaw swelling was measured for all four paws in duplicate for each mouse. A statistically significant reduction in foot paw swelling was observed in mice consuming Bifidobacterium 35624 but not with Lactobacillus 433118 (FIG. 9). This study demonstrates that this probiotic bacterium induces immune-regulatory cells and mediators outside the gastrointetsinal tract. The most important cellular mediator of these effects are dendritic cells and the regulatory T cells stimulated by dendritic cells.

### EXAMPLE 6

### Anti-cancer Properties of Bacterial Stimulated Dendritic Cells

### Method

Bone marrow derived dendritic cells were isolated from Balb/c mice using magnetic bead isolation and cultured for 7-8 days in vitro in the presence of GM-CSF and IL-4. Following expansion and maturation, dendritic cells were incubated with or without Bifidobacterium infantis 35624 for 90 minutes, in addition to co-incubation with JBS tumour cell lysates. JBS tumour cells survive and proliferate rapidly in immune competent balb/c mice. Balb/c mice were injected subcutaneous with:

Group 1: 1×10⁵ dendritic cells pre-incubated with JBS lysates alone;

Group 2: 1×10⁵ dendritic cells pre-incubated with JBS lysates plus Bifidobacterium 35624.

The balb/c mice were injected on two separate occasions using the procedure outlined above (n=8 mice per group). Concurrently, all mice were injected with live JBS tumour cells. Two weeks following tumour inoculation, all mice were sacrificed by cervical dislocation, tumours excised and weighed.

### Results

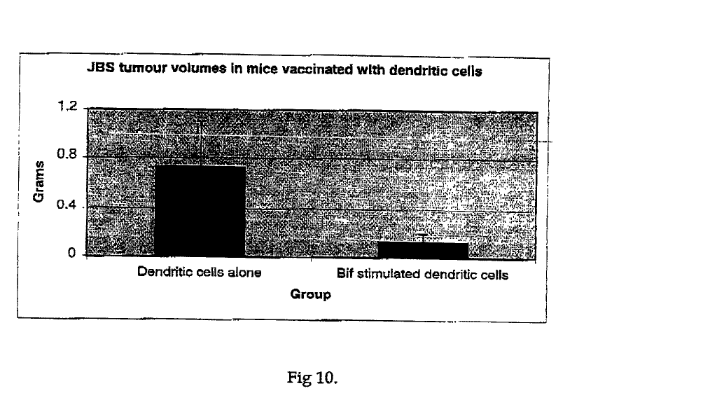

The mean tumour volume was decreased in mice vaccinated with Bifidobacterium stimulated dendritic cells compared to mice vaccinated by dendritic cells alone (FIG. 10). Thus, adoptive transfer of Bifidobacterium 35624 activated dendritic cells can restrict the rate of JBS tumour growth.

The complexity and intimacy of the interactions that occur between bacteria and the host eukaryotic cells have only begun to be elucidated. The nature of these interactions creates a major paradox. The human being has a vast number of bacteria living on or in the host, representing 90% of all cells found in the body. These bacteria constitute the commensal flora found on all mucosal and epidermal structures. Populations of these bacteria vary between the oral cavity, gastrointestinal tract, urogenital tract and the skin surface. The immune system recognizes the presence of these foreign microbes and therefore would be expected to launch significant immune responses resulting in chronic inflammatory lesions at these sites. However, this is not the case. The commensal microflora and the host systems exist in a finely balanced environment whereby bacterial communities thrive and host tissues are not damaged by their own immune system. Evolution has selected for individuals whose immune system tolerates the presence of the nonpathogenic commensal flora while being able to react rapidly to the presence of pathogenic microbes. While the mechanisms underlying this immunological perception are currently unclear, dendritic cells appear to have the ability to secrete different cytokines depending on the specific bacterial stimulus. As the dendritic cell provides the link between innate and adaptive immune responses, it is perfectly poised to control the nature of this response. Ultimately, the decision to attack or tolerate specific antigens may reside with the dendritic cell.

This invention is not limited to dendritic cells isolated only in the manner as described herein, but applies to dendritic cells isolated using any technology and derived from any body compartment or tissue.

This invention describes the cytokine network established due to stimulation of dendritic cells with Lactobacillus, Bifidobacterium and Salmonella species. However, this technology can be applied to all bacterial types and should not be limited to these bacterial strains alone. It is expected that stimulation of dendritic cells with different bacterial species will result in dendritic cells with different cytokine profiles. These different immuno-therapeutic properties are applicable to a wide range of disease states.

It is unknown whether the bacterial strains are required to exert an immuno-modulatory effect or if individual active components of the bacterial strains can be utilised alone. Proinflammatory components of certain bacterial strains have been identified. The proinflammatory effects of gram-negative bacteria are mediated by liposaccharide (LPS). LPS alone induces a proinflammatory network, partially due to LPS binding to the CD14 receptor on monocytes. It is assumed that components of probiotic bacteria possess anti-inflammatory activity, due to the effects of the whole cells. Upon isolation of these components, pharmaceutical grade manipulation is anticipated. Therefore the term bacterial strain as used in this specification refers to active components thereof.

The general use of the bacterial strains is in the form of viable cells. However, it can also be extended to non-viable cells such as killed cultures or compositions containing beneficial factors expressed by the bacterial strains. This could include thermally killed micro-organisms or micro-organisms killed by exposure to altered pH or subjection to pressure. With non-viable cells product preparation is simpler, cells may be incorporated easily into pharmaceuticals and storage requirements are much less limited than viable cells. Lactobacillus casei YIT 9018 offers an example of the effective use of heat killed cells as a method for the treatment and/or prevention of tumour growth as described in U.S. Pat. No. 4,347,240.

The specific application of bacterial activated dendritic cells for the treatment of human disease will depend on the disease state being treated. Dendritic cells can be isolated from all types of human tissue, including peripheral blood, mucosal sites, etc. It is envisaged that tissue will be isolated from a patient by a physician. Following removal of patient tissue, dendritic cells are purified, under sterile conditions, using antibody-labelling techniques (such as magnetic bead isolation). Dendritic cells may be cultured in vitro with cytokines and subsequently activated by bacterial cells, or can be activated immediately following purification by bacterial cells. Bacterial activated dendritic cells are administered back to the same patient from whom they were first isolated. The route of administration may be parenteral or enteral, including subcutaneous injection, intramuscular injection, intraperitoneal injection, intravenous injection, intravenous drip, nasal spray, oral consumption in enteric coated capsules, etc. Dendritic cells may be administered in a saline or nutrient solution, or can be administered with an adjuvant. For treatment of cancer patients, dendritic cells can be co-administered with tumour cells, preferably derived from the same patient. In other disease states, dendritic cells may be co-administered with antigens associated with disease pathology, such as myelin basic protein (i.e. multiple sclerosis). It is anticipated that dendritic cells may be administered at greater than 1×10⁵ cells per patient and that treatment can be repeated as required.

The invention is not limited to the embodiments hereinbefore described which may be varied in detail.

### References

Wilmes-Riesenberg M. R., Bearson B., Foster J. W. & Curtiss R. Role of the acid tolerance response in virulence of Salmonella typhimurium. Infect. Immun., 1996:1085-92.

Seder R A, Paul W E, Davis M M, Fazekas de St Groth B. The presence of interleukin 4 during in vitro priming determines the lymphokine-producing potential of CD4+T cells from T cell receptor transgenic mice. J Exp Med 1992 Oct 1;176(4):1091-8.

Mosmann T. R. & Sad S. The expanding universe of T-cell subsets: Th1, Th2 and more. Immunol. Today, 1996; 17:138-46.

Trinchieri G, Peritt D, Gerosa F. Acute induction and priming for cytokine production in lymphocytes. Cytokine Growth Factor Rev 1996 August;7(2):123-32.

Maldonado-Lopez R., De Smedt T., Michel P., Godfroid J., Pajak B., Heirman C., Thielemans K., Leo O., Urbain J. & Moser M. CD8□+ and CD8□− subclasses of dendritic cells direct the development of distinct T helper cells in vivo. J. Exp. Med., 1999; 189:587-92.

Groux H., O'Garra A., Bigler M., Rouleau M., Antonenko S., de Vries J. E. & Roncarolo M. G. A CD4+ T-cell subset inhibits antigen-specific T-cell responses and prevents colitis. Nature, 1997; 389:737-42.

Jonuleit H., Schmitt E., Schuler G., Knop J. & Enk A. H. Induction of interleukin 10-producing, nonproliferating CD4+ T cells with regulatory properties by repetitive stimulation with allogeneic immature human dendritic cells. J. Exp. Med., 2000; 192:1213-22.

Kannourakis G, Abbas A. The role of cytokines in the pathogenesis of Langerhans cell histiocytosis. Br J Cancer Suppl 1994 September;23:537-40.

Kouskoff V, Korganow A S, Duchatelle V, Degott C, Benoist C, Mathis D. Organ-specific disease provoked by systemic autoimmunity. Cell Nov. 29, 1996;87(5):811-22.

Roubenoff R, Roubenoff R A, Cannon J G, Kehayias J J, Zhuang H, Dawson-Hughes B, Dinarello C A, Rosenberg I H. Rheumatoid cachexia: cytokine-driven hypermetabolism accompanying reduced body cell mass in chronic inflammation. J Clin Invest 1994 June;93(6):2379-86.

Kwiatkowski D, Hill A V, Sambou I, Twumasi P, Castracane J, Manogue K R, Cerami A, Brewster D R, Greenwood B M. TNF concentration in fatal cerebral, non-fatal cerebral, and uncomplicated Plasmodium falciparum malaria. Lancet Nov. 17, 1990;336(8725):1201-4.

McGuire W, Hill A V, Allsopp C E, Greenwood B M, Kwiatkowski D. Variation in the TNF-alpha promoter region associated with susceptibility to cerebral malaria. Nature Oct. 6, 1994;371(6497):508-10.

Stuber F, Petersen M, Bokelmann F, Schade U. A genomic polymorphism within the tumor necrosis factor locus influences plasma tumor necrosis factor-alpha concentrations and outcome of patients with severe sepsis. Crit Care Med 1996 March;24(3):381-4.

Dinarello C A. The role of interleukin-1 in host responses to infectious diseases. Infect Agents Dis 1992 October;1(5):227-36.

Pfeffer K, Matsuyama T, Kundig T M, Wakeham A, Kishihara K, Shahinian A, Wiegmann K, Ohashi P S, Kronke M, Mak T W. Mice deficient for the 55 kd tumor necrosis factor receptor are resistant to endotoxic shock, yet succumb to L. monocytogenes infection. Cell May 7, 1993;73(3):457-67.

Moreland L W, Baumgartner S W, Schiff M H, Tindall E A, Fleischmann R M, Weaver A L, Ettlinger R E, Cohen S, Koopman W J, Mohler K, Widmer M B, Blosch C M. Treatment of rheumatoid arthritis with a recombinant human tumor necrosis factor receptor (p75)-Fc fusion protein. N Engl J Med Jul. 17,1997;337(3):141-7.

Stack W A, Mann S D, Roy A J, Heath P, Sopwith M, Freeman J, Holmes G, Long R, Forbes A, Kamm M A. Randomised controlled trial of CDP571 antibody to tumour necrosis factor-alpha in Crohn's disease. Lancet Feb. 22,1997;349(9051):521-4.

Neurath M F, Pettersson S, Meyer zum Buschenfelde K H, Strober W. Local administration of antisense phosphorothioate oligonucleotides to the p65 subunit of NF-kappa B abrogates established experimental colitis in mice. Nat Med 1996 September;2(9):998-1004.

Asserman C., Mauze S., Leach M. W., Coffman R. L. & Powrie F. An essential role for interleukin 10 in the function of regulatory T cells that inhibit intestinal inflammation. J. Exp. Med., 1999; 190:995-1003.

Drakesmith H., Chain B. & Beverly B. How can dendritic cells cause autoimmune disease? Immunol. Today, 2000; 21:214-7.

Link H., Huang Y. -M., Masterman T. & Xiao B. -G. Vaccination with autologous dendritic cells: from experimental autoimmune encephalomyelitis to multiple sclerosis. J. Neuroimmunol., 2001; 114:1-7.

Huang Y. M., Yang J. S., Xu L. Y., Link H. & Xiao B. G. Autoantigen-pulsed dendritic cells induce tolerance to experimental allergic encephalomyelitis in Lewis rats. Clin. Exp. Immunol., 2000; 122, 437-44.

Papaccio G., Nicoletti F., Pisanti F. A., Bendtzen K. & Galdieri M. Prevention of spontaneous autoimmune diabetes in NOD mice by transferring in vitro antigenpulsed syngeneic dendritic cells. Endocrinology, 2000; 141:1500-5.

Thorbecke G. J., Umetsu D. T., deKruyff R. H., Hansen G., Chen L. Z. & Hochwald G. M. When engineered to produce TGF-□1, antigen specific T cells down regulate Th1 cell-mediated autoimmune and Th2 cell-mediated allergic inflammatory processes. Cytokine Growth Factor Rev., 2000; 11:89-96.

Wilson J. M. Researchers and regulators reflect on first gene therapy death. Nat. Med., 2000; 6:6.

Restifo N P, Kawakami Y, Marincola F, Shamamian P, Taggarse A, Esquivel F, Rosenberg S A. Molecular mechanisms used by tumors to escape immune recognition: immunogenetherapy and the cell biology of major histocompatibility complex class I. J Immunother 1993 October;14(3):182-90.

Cromme F V, Airey J, Heemels M T, Ploegh H L, Keating P J, Stem P L, Meijer C J, Walboomers J M. Loss of transporter protein, encoded by the TAP-1 gene, is highly correlated with loss of HLA expression in cervical carcinomas. J Exp Med Jan.1, 1994;179(1):335-40.

Shu S, Plautz G E, Krauss J C, Chang A E. Tumor immunology. JAMA Dec. 10, 1997 ;278(22):1972-81.

Staveley-O' Carroll K, Sotomayor E, Montgomery J, Borrello I, Hwang L,

Fein S, Pardoll D, Levitsky H. Induction of antigen-specific T cell anergy: An early event in the course of tumor progression. Proc Natl Acad Sci USA Feb. 3, 1998 ;95(3):1178-83.

Tsang K Y, Zaremba S, Nieroda C A, Zhu M Z, Hamilton J M, Schlom J. Generation of human cytotoxic T cells specific for human carcinoembryonic antigen epitopes from patients immunized with recombinant vaccinia-CEA vaccine. J Natl Cancer Inst Jul. 5, 1995;87(13):982-90.

Hahne M, Rimoldi D, Schroter M, Romero P, Schreier M, French L E, Schneider P, Bornand T, Fontana A, Lienard D, Cerottini J, Tschopp J. Melanoma cell expression of Fas(Apo-1/CD95) ligand: implications for tumor immune escape. Science Nov. 22, 1996; 274(5291):1363-6.

Gahan C G, Barrett J R, O'Brien M G, O'Sullivan G C, Shanahan F, Collins J K. Innate resistance to Listeria monocytogenes in tumor-bearing mice. J Leukoc Biol 1997 December;62(6):726-32.

O'Sullivan G C, Corbett A R, Shanahan F, Collins J K. Regional immunosuppression in esophageal squamous cancer: evidence from functional studies with matched lymph nodes. J Immunol Nov. 15, 1996;157(10):4717-20.

Spellman J E, Gollnick S O, Zhang P J, Tomasi T B. Cytokine production by human soft tissue sarcomas: implications for immunosuppression within the tumour bed. Surg Oncol 1996 October-December;5(5-6):237-44.

O'Mahony A M, O'Sullivan G C, O'Connell J, Cotter T G, Collins J K. An immune suppressive factor derived from esophageal squamous carcinoma induces apoptosis in normal and transformed cells of lymphoid lineage. J Immunol Nov.1, 1993 ;151(9):4847-56.

Nestle F., Alijagic S., Gilliet M., Sun Y., Grabbe S., Dummer R., Burg G. & Schadendorf D. Vaccination of melanoma patients with peptide- or tumor lysate-pulsed dendritic cells. Nat. Med., 1998; 2:328-32.

Walker R. J. Vaccine, 1994, 12, 387. Walker R .J. Vaccine, 1994, 12, 387.

Roberts A B, Flanders K C, Heine U I, Jakowlew S, Kondaiah P, Kim S J, Sporn M B. Transforming growth factor-beta: multifunctional regulator of differentiation and development. Philos Trans R Soc Lond B Biol Sci 1990 Mar 12;327(1239):145-54.

Wahl S M, Allen J B, Weeks B S, Wong H L, Klotman P E. Transforming growth factor beta enhances integrin expression and type IV collagenase secretion in human monocytes. Proc Natl Acad Sci USA May 15, 1993;90(10):4577-81.

Ding A, Nathan C F, Graycar J, Derynck R, Stuehr D J, Srimal S. Macrophage deactivating factor and transforming growth factors-beta 1 -beta 2 and -beta 3 inhibit induction of macrophage nitrogen oxide synthesis by IFN-gamma. J Immunol Aug. 1, 1999;145(3):940-4.

Arai K I, Lee F, Miyajima A, Miyatake S, Arai N, Yokota T. Cytokines: coordinators of immune and inflammatory responses. Annu Rev Biochem 1990;59:783-836.

Aiello F B, Longo D L, Overton R, Takacs L, Durum S K. A role for cytokines in antigen presentation: IL-1 and IL-4 induce accessory functions of antigen-presenting cells. J Immunol Apr. 1, 1990;144(7):2572-81.

Corcoran M L, Stetler-Stevenson W G, Brown P D, Wahl L M. Interleukin 4 inhibition of prostaglandin E2 synthesis blocks interstitial collagenase and 92-kDa type IV collagenase/gelatinase production by human monocytes. J Biol Chem Jan. 5, 1992 ;267(1):515-9.

Mangan D F, Welch G R, Wahl S M. Lipopolysaccharide, tumor necrosis factor-alpha, and IL-1 beta prevent programmed cell death (apoptosis) in human peripheral blood monocytes. J immunol Mar. 1, 1991;146(5):1541-6.

Hart P H, Ahem M J, Smith M D, Finlay-Jones J J. Regulatory effects of IL-13 on synovial fluid macrophages and blood monocytes from patients with inflammatory arthritis. Clin Exp Immunol 1995 March;99(3):331-7.

de Waal Malefyt R, Haanen J, Spits H, Roncarolo M G, te Velde A, Figdor C, Johnson K, Kastelein R, Yssel H, de Vries J E. Interleukin 10 (IL-10) and viral IL-10 strongly reduce antigen-specific human T cell proliferation by diminishing the antigen-presenting capacity of monocytes via downregulation of class II major histocompatibility complex expression. J Exp Med Oct 1, 1991;174(4):915-24.

Go N F, Castle B E, Barrett R, Kastelein R, Dang W, Mosmann T R, Moore K W, Howard M. Interleukin 10, a novel B cell stimulatory factor: unresponsiveness of X chromosome-linked immunodeficiency B cells. J Exp Med Dec. 1,1990;172(6):1625-31.

Mertz P M, DeWitt D L, Stetler-Stevenson W G, Wahl L M. Interleukin 10 suppression of monocyte prostaglandin H synthase-2. Mechanism of inhibition of prostaglandin-dependent matrix metalloproteinase production. J Biol Chem Aug. 19, 1994;269(33):21322-9.

Ma X, Aste-Amezaga M, Gri G, Gerosa F, Trinchieri G. Immunomodulatory functions and molecular regulation of IL-12. Chem Immunol 1997;68:1-22.

Schmitt E, Rude E, Germann T. The immunostimulatory function of IL-12 in T-helper cell development and its regulation by TGF-beta, IFN-gamma and IL-4. Chem Immunol 1997;68:70-85.

D'Andrea A, Ma X, Aste-Amezaga M, Paganin C, Trinchieri G. Stimulatory and inhibitory effects of interleukin (IL)-4 and IL-13 on the production of cytokines by human peripheral blood mononuclear cells: priming for IL-12 and tumor necrosis factor alpha production. J Exp Med Feb. 1, 1995;181(2):537-46.

Leonard J P, Waldburger K E, Schaub R G, Smith T, Hewson A K, Cuzner M L, Goldman S J. Regulation of the inflammatory response in animal models of multiple sclerosis by interleukin-12. Crit Rev Immunol 1997;17(5-6):545-53.

Donnelly R P, Fenton M J, Finbloom D S, Gerrard T L. Differential regulation of IL-1 production in human monocytes by IFN-gamma and IL-4. J Immunol Jul. 15, 1990;145(2):569-75.

Wahl L M, Corcoran M E, Mergenhagen S E, Finbloom D S. Inhibition of phospholipase activity in human monocytes by IFN-gamma blocks endogenous prostaglandin E2-dependent collagenase production. J Immunol May. 1, 1990;144(9):3518-22.

Neale T J, Ruger B M, Macaulay H, Dunbar P R, Hasan Q, Bourke A, Murray-McIntosh R P, Kitching A R. Tumor necrosis factor-alpha is expressed by glomerular visceral epithelial cells in human membranous nephropathy. Am J Pathol 1995 June;146(6):1444-54.

Schall T J, Lewis M, Koller K J, Lee A, Rice G C, Wong G H, Gatanaga T, Granger G A, Lentz R, Raab H, et al. Molecular cloning and expression of a receptor for human tumor necrosis factor. Cell Apr. 20, 1990;61(2):361-70.

Wong G H, Goeddel D V. Tumour necrosis factors alpha and beta inhibit virus replication and synergize with interferons. Nature Oct. 30-Nov. 5, 1986;323(6091):819-22.

Livingston D H, Appel S H, Sonnenfeld G, Malangoni M A. The effect of tumor necrosis factor-alpha and interferon-gamma on neutrophil function. J Surg Res 1989 April;46(4):322-6.

Cicco N A, Lindemann A, Content J, Vandenbussche P, Lubbert M, Gauss J, Mertelsmann R, Herrmann F. Inducible production of interleukin-6 by human polymorphonuclear neutrophils: role of granulocyte-macrophage colony-stimulating factor and tumor necrosis factor-alpha. Blood May 15, 1990;75(10):2049-52.

Mangan D F, Welch G R, Wahl S M. Lipopolysaccharide, tumor necrosis factor-alpha, and IL-1 beta prevent programmed cell death (apoptosis) in human peripheral blood monocytes. J Immunol Mar. 1, 1991;146(5):1541-6.

Dinarello C A, Cannon J G, Wolff S M. New concepts on the pathogenesis of fever. Rev Infect Dis 1988 January-February;10(1):168-89.

1. Dendritic cells which have been exposed to at least one bacterial strain.

2. Dendritic cells which have been exposed to bacterial species present in the human commensal flora.

3. Dendritic cells as claimed in claim 1 wherein the bacterial strain is a Lactobacillus.

4. Dendritic cells as claimed in claim 3 wherein the Lactobacillus is Lactobacillus salivarius.

5. Dendritic cells as claimed in claim 4 wherein the Lactobacillus is Lactobacillus salivarius subspecies salivarius.

6. Dendritic cells as claimed in claim 1 wherein the bacterial strain is Lactobacillus salivarius subspecies salivarius 433118.

7. Dendritic cells as claimed in claim 1 wherein the bacterial strain is a Bifidobacterium.

8. Dendritic cells as claimed in claim 7 wherein the bacterial strain is Bifidobacterium infantis.

9. Dendritic cells as claimed in claim 7 wherein the bacterial strain is Bifidobacterium infantis 35624.

10. Dendritic cells as claimed in claim 1 wherein the bacterial strain is salmonella.

11. Dendritic cells as claimed in claim 10 wherein the bacterial strain is Salmonella typhimurium.

12. Dendritic cells as claimed in claim 10 wherein the bacterial strain is Salmonella typhimurium UK1.

13. Dendritic cells as claimed in claim 1 exposed to dead bacteria, or components or mutants thereof.

14. An active derivative, fragment or mutant of dendritic cells as claimed in claim 1.

15. A formulation comprising dendritic cells as claimed in claim 1 or an active derivative, fragment or mutant thereof.

16. A pharmaceutical comprising dendritic cells as claimed in claim 1 or an active derivative, fragment or mutant thereof.

17. A vaccine comprising dendritic cells as claimed in claim 1 or an active derivative, fragment or mutant thereof.

18. A method for activating dendritic cells comprising exposing dendritic cells to at least one bacterial strain.

19. A method for activating dendritic cells comprising exposing dendritic cells to bacterial species present in the human commensal flora.

20. A method for the prophylaxis and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising administering dendritic cells as claimed in claim 1 or an active derivative, fragment or mutant thereof.
